(19) United States
(12) Patent Application Publication
(10) Pub. No.: US 2021/0084989 A1
(43) Pub. Date: Mar. 25, 2021

(54) MODULAR SYSTEM FOR VAPORIZING LIQUID VAPORIZING MATERIAL

(71) Applicant: Mya Johnson, Atlantis, FL (US)

(72) Inventor: Mya Johnson, Atlantis, FL (US)

(21) Appl. No.: 16/576,300

(22) Filed: Sep. 19, 2019

(51) Int. Cl. A24F 47/00; A61M 11/04

(52) U.S. Cl. CPC A24F 47/008; A24B 15/167; A61M 11/042

(57) ABSTRACT

A modular system for vaporizing liquid vaporizing material is disclosed. The modular system includes a mouthpiece having at least one mouthpiece inlet on a mouthpiece sidewall. A first housing is configured to removably attach to the mouthpiece. The first housing includes a tube, an oil chamber, and a coil unit configured to be removably attachable within a coil unit compartment of the first housing. The coil unit includes, a mouth, at least one coil unit passageway, a heating element, a pair of first conductors, and a wick. A second housing having at least one second housing inlet on a second housing sidewall is configured to removably attach with the first housing. The second housing includes a central processing unit, a power source, a display, at least one control, a charging port, and a pair of second conductors configured to contact with the first set of conductors.

### CROSS-REFERENCE TO RELATED APPLICATIONS

Not applicable.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to the field of electronic smoking, and more specifically to the field of electronic cigarettes and personal vaporizers.

### BACKGROUND

Since entering the U.S. market in 2007, electronic cigarettes, also known as e-cigarettes, e-cigs, electronic nicotine delivery systems, vaporizer cigarettes, or vape pens, have attracted a growing supply of consumers, and sales are expected to increase in the years ahead. An electronic cigarette is a handheld battery-powered vaporizer that simulates smoking by providing some of the behavioral aspects of smoking, including the hand-to-mouth action of smoking, but without combusting tobacco. Using an electronic cigarette is known as “vaping” and the user is referred to as a “vaper.” Electronic cigarettes typically have a heating element that atomizes a liquid solution called e-liquid. E-liquids typically include nicotine, flavoring and a humectant, such as propylene glycol, to retain moisture and create an aerosol when heated. Electronic cigarettes are automatically activated by taking a puff or pressing a button. Some e-cigarettes look like traditional cigarettes, but they come in many variations. Most versions are reusable, though some are disposable.

Aerosol production generally involves three stages: preprocessing, aerosol generation and postprocessing. The first stage involves the transport of the e-liquid to the aerosol generator. Capillary action through a wick is the primary means used by the majority of current electronic cigarettes to control the delivery of e-liquid to the aerosol generator. The second stage of aerosol processing involves aerosol generation, in which the e-liquid comes in contact with the heating element as described above. The final stage of aerosol processing occurs as the aerosol travels through the central air passage to the consumer.

As the electronic cigarette industry continues to evolve, new products are quickly developed and brought to market. Although numerous disposable and reusable electronic cigarette product options exist, the technology is still young, and largely based on repurposed electronic cigarette hardware that has left consumers plagued by a familiar suite of problems. Specifically, currently marketed electronic cigarettes lack interchangeability of components and configurations that determine the airflow rate and heating element temperature. For example, the heating element's resistance, material, and the voltage across it determine the current flow and element temperature. The heating element temperature and temperature duration influence the aerosol properties. As a result, user modification is of particular concern since the performance, risks and safety associated with a particular brand's configuration might change significantly when that configuration is modified.

Therefore, a need exists to improve over the prior art and more particularly, for a modular system for vaporizing liquid vaporizing material that supports interchangeability of components feature adjustments that determine the airflow rate and heating element temperature.

### SUMMARY

A modular system for vaporizing liquid vaporizing material is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a modular system for vaporizing liquid vaporizing material is disclosed. The modular system includes a mouthpiece having at least one mouthpiece inlet on a mouthpiece sidewall and an air outlet at a mouthpiece end. The system further includes a first housing that is configured to removably attach to the mouthpiece. The first housing includes a tube that is disposed within the first housing such that a first end of the tube is proximate to a first end of the first housing. An oil chamber within the first housing is configured for storing liquid vaporizing material. A coil unit is configured to be removably attachable within a coil unit compartment of the first housing. The coil unit includes a mouth at a first end of the coil unit, at least one coil unit passageway within the coil unit, a heating element, a pair of first conductors for providing electrical communication between the heating element and a power source externally located from the coil unit, and a wick in contact with the heating element and positioned with a heating chamber. The system further includes a second housing having a first end and a second end and having at least one second housing inlet on a second housing sidewall. The first end of the second housing is configured to removably attach with a second end of the first housing. The second housing includes a central processing unit, the power source, a display, at least one control for controlling the system, a charging port, and a pair of second conductors configured to contact and provide electrical communication with the first set of conductors.

In another embodiment, a modular system for vaporizing liquid vaporizing material is disclosed. The system includes a mouthpiece having an air outlet at a mouthpiece end. The system also includes a first housing attached to the mouthpiece. The first housing includes a tube disposed within the first housing, and an oil chamber for storing liquid vaporizing material, and a coil unit within a coil unit compartment of the first housing. The coil unit includes a mouth at a first end of the coil unit, at least one coil unit passageway within the coil unit, a heating element, a pair of first conductors for providing electrical communication between the heating element and a power source externally located from the coil unit, and a wick in contact with the heating element and positioned with a heating chamber. The system further includes a second housing that is configured to removably attach with a second end of the first housing. The second housing includes a central processing unit, the power source, a display, at least one control for controlling the system, a charging port, and a pair of second conductors configured to contact and provide electrical communication with the first set of conductors.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is an exploded perspective view of a modular system for vaporizing liquid vaporizing material, according to an example embodiment of the present invention;

FIG. 2A is a cross-sectional front view of an airflow path for a first housing, according to a example embodiment of the present invention;

FIG. 2B is a cross-sectional side view of an airflow path for a first housing, according to an example embodiment of the present invention;

FIG. 2C is a cross-sectional bottom view of a coil unit passageway opening located within a coil unit, according to an example embodiment of the present invention;

FIG. 3A is a front view of a first housing attached to a mouthpiece, according to an example embodiment of the present invention;

FIG. 3B is a left side view of a first housing attached to a mouthpiece, according to an example embodiment of the present invention;

FIG. 3C is a bottom view of a first housing attached to a mouthpiece, according to an example embodiment of the present invention;

FIG. 3D is a front view of the interior components of a first housing, according to an example embodiment of the present invention;

FIG. 3E is a side view of the interior components of a first housing, according to an example embodiment of the present invention;

FIG. 4 is an elevated partially exploded perspective view of a coil unit and a silicone ring, according to an example embodiment of the present invention;

FIG. 5 is an elevated front perspective view of a silicone ring positioned on a coil unit, according to an example embodiment of the present invention;

FIG. 6 is an elevated front perspective view of a heating element coiled around a wick, according to an example embodiment of the present invention;

FIG. 7 is an elevated front perspective view of a heating element coiled around a wick and positioned on a coil unit, according to an example embodiment of the present invention;

FIG. 8 is an elevated partially exploded perspective view of a coil unit and a pair of silicone sleeves, according to an example embodiment of the present invention;

FIG. 9 is an elevated partially exploded perspective view of a coil unit and a pair of first conductors, according to an example embodiment of the present invention;

FIG. 10 is an elevated bottom perspective view of a coil unit, wherein a pair of silicone sleeves and a pair of first conductors are positioned inside the coil unit, according to an example embodiment of the present invention;

FIG. 11 is an elevated partially exploded perspective view of a mouth that is configured to attach to a first end of a coil unit, according to an example embodiment of the present invention;

FIG. 12 is an elevated front perspective view of an assembled coil unit, according to an example embodiment of the present invention;

FIG. 13 is an elevated partially exploded perspective view of a tube and a first housing, according to an example embodiment of the present invention;

FIG. 14 is a cross-sectional front view of a tube disposed within a first housing; according to an example embodiment of the present invention;

FIG. 15 is an elevated partially exploded perspective view of a first housing and a coil unit, according to an example embodiment of the present invention;

FIG. 16 is a cross-sectional front view of a coil unit disposed within a first housing; according to an example embodiment of the present invention;

FIG. 17 is an elevated partially exploded perspective view of a first housing and a first housing sealing cap, according to an example embodiment of the present invention;

FIG, 18 is an elevated front perspective view of a first housing sealing cap attached to a first end of a first housing; according to an example embodiment of the present invention;

FIG. 19 is an elevated partially exploded perspective view of a first housing and a mouthpiece, according to an example embodiment of the present invention;

FIG. 20 is an elevated front perspective view of a mouthpiece attached to a first end of a first housing; according to an example embodiment of the present invention;

FIG. 21 is an elevated partially exploded perspective view of a mouthpiece cover and a first housing attached to a mouthpiece, according to an example embodiment of the present invention;

FIG. 22 is an elevated front perspective view of a mouthpiece cover attached on a mouthpiece, according to an example embodiment of the present invention;

FIG. 23 is an elevated partially exploded perspective view of a first housing cover and a first housing attached to a mouthpiece, wherein a mouthpiece cover is attached on the mouthpiece, according to an example embodiment of the present invention;

FIG. 24A is an elevated bottom perspective view of a first housing attached to a mouthpiece, wherein a mouthpiece cover is attached on the mouthpiece, and a first housing cover is attached on a bottom end of the first housing, according to an example embodiment of the present invention;

FIG. 24B is an elevated front perspective view of a first housing attached to a mouthpiece, wherein a mouthpiece cover is attached on the mouthpiece, and a first housing cover is attached on a bottom end of the first housing, according to an example embodiment of the present invention;

FIG. 25A is a cross-sectional front view of a second housing, according to an example embodiment of the present invention;

FIG. 25B is a cross-sectional side view of a second housing, according to an example embodiment of the present invention;

FIG. 26 is a partially exploded bottom view of a central processing unit and a power source, according to an example embodiment of the present invention;

FIG. 27 is an elevated side perspective view of a central processing unit connected to a power source, according to an example embodiment of the present invention;

FIG. 28 is an elevated partially exploded perspective view of an inner frame and a central processing unit connected to a power source, according to an example embodiment of the present invention;

FIG. 29 is an elevated bottom perspective view of a central processing unit connected to a power source, wherein the central processing unit and the power source are positioned within an inner frame, according to an example embodiment of the present invention;

FIG. 30 is an elevated partially exploded perspective view of an end cap and a central processing unit and power source positioned within an inner frame, according to an example embodiment of the present invention;

FIG. 31 is an elevated bottom perspective view of an end cap attached on a first end of an inner frame, according to an example embodiment of the present invention;

FIG. 32 is an elevated partially exploded perspective view of a second housing and an inner frame, wherein the central processing unit and power source are positioned within the inner frame and an end cap is attached on a first end of the inner frame; according to an example embodiment of the present invention;

FIG. 33 is an elevated front perspective view of an inner frame inserted into a second housing, according to an example embodiment of the present invention;

FIG. 34 is an elevated partially exploded perspective view of a button for controlling the system and a button holder, according to an example embodiment of the present invention;

FIG. 35 is an elevated front perspective view of a button for controlling the system positioned on a button holder, according to an example embodiment of the present invention;

FIG. 36 is an elevated partially exploded perspective view of a button for controlling the system positioned on a button holder and an inner frame inserted into a second housing, according to an example embodiment of the present invention;

FIG. 37 is an elevated front perspective view of a button for controlling the system positioned inside a circular shaped opening located on the surface of a second housing, according to an example embodiment of the present invention;

FIG. 38 is an elevated partially exploded perspective view of a display cover and an inner frame inserted into a second housing, according to an example embodiment of the present invention;

FIG. 39 is an elevated front perspective view of a display cover positioned inside a rectangular shaped opening located on the surface of a second housing, according to an example embodiment of the present invention;

FIG. 40A is a front view of an assembled second housing, according to an example embodiment of the present invention;

FIG. 40B is a right side view of an assembled second housing, according to an example embodiment of the present invention;

FIG. 40C is a top view of an assembled second housing, according to an example embodiment of the present invention;

FIG. 40D is a bottom view of an assembled second housing, according to an example embodiment of the present invention;

FIG. 41 is an elevated front perspective view of a first housing attached to a mouthpiece, wherein a mouthpiece cover is attached on the mouthpiece, and a first housing cover is attached on a bottom end of the first housing, and an assembled second housing, according to an example embodiment of the present invention;

FIG. 42 is an elevated front perspective view of a first housing attached to a mouthpiece being inserted into an assembled second housing, according to an example embodiment of the present invention;

FIG. 43A is a front view of a modular system for vaporizing liquid vaporizing material, according to an example embodiment of the present invention;

FIG. 43B is a rear view of a modular system for vaporizing liquid vaporizing material, according to an example embodiment of the present invention;

FIG. 43C is a right side view of a modular system for vaporizing liquid vaporizing material, according to an example embodiment of the present invention;

FIG. 43D is a left side view of a modular system for vaporizing liquid vaporizing material, according to an example embodiment of the present invention;

FIG. 43E is a top view of a modular system for vaporizing liquid vaporizing material, according to an example embodiment of the present invention;

FIG. 43F is a bottom view of a modular system for vaporizing liquid vaporizing material, according to an example embodiment of the present invention;

FIG. 44 is a block diagram showing the main components of a modular system for vaporizing liquid vaporizing material, according to an example embodiment of the present invention; and,

FIG. 45 is a block diagram of a system including an example central processing unit, according to an example embodiment of the present invention.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The present invention improves upon the prior art by providing multiple air inlets to help facilitate the flow of air through the coil and condense the e-liquid into vapor, which results in greater flavor and vapor production. Specifically, the modular system includes a mouthpiece having at least one mouthpiece inlet on a mouthpiece sidewall and a second housing having at least one second housing inlet on a second housing sidewall. When there is an increase of air flowing through the coil, the vapor gets less dense and this produces large clouds. This also means that because the vapor is less concentrated, the impact on the throat is reduced, giving a smoother taste. In addition, the increase of air flowing through the coil produces cooler vapor and an airier draw.

The present invention also improves upon the prior art by including interchangeable parts to enable users to modify the character of the modular system. Specifically, the modular system includes a first housing configured to removably attach to the mouthpiece, a coil unit configured to be removably attachable within a coil unit compartment of the first housing, and a second housing configured to removably attach with the second end of the first housing. For example, different wicks have properties that determine how well the flavor comes through and how quickly the liquid vaporizing material can flow into the atomizer. Although many coil units use a cotton wick as their heating element, a user may want to utilize a coil unit containing a ceramic wick for cannabis oil because it can withstand the high temperatures needed to vaporize cannabis oil without burning the material. Additionally, a user may clean and refill the first housing with a variety of liquid vaporizing materials by removing the coil unit from the coil unit compartment of the first housing.

The present invention further improves upon the prior art by providing a display to show operating state parameters, such as battery life, voltage, average use cycle, and safety warnings. Specifically, the display of the modular system is configured to display a graphical representation of at least an amount of charge in the power source and a voltage at which power is provided from the power source.

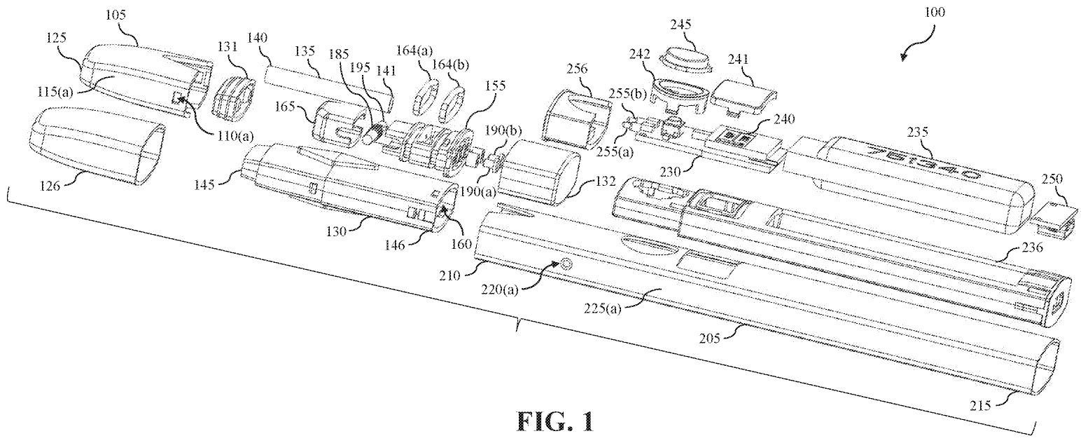

Referring now to the Figures, FIG. 1 is an exploded perspective view of a modular system 100 for vaporizing liquid vaporizing material, according to an example embodiment of the present invention. The modular system 100 includes a mouthpiece 105 comprising a hollow rectangular shaped body with a frontward facing side 106, a rearward facing side 107, two opposing sidewalls 115(a), 115(b), an end wall 108, and a central opening 109 located on a bottom end 111 of the mouthpiece 105. The mouthpiece 105 is the part of the modular system 100 from which a user places to their mouth to inhale vapor. In the present embodiment, the mouthpiece 100 includes a triangular shaped cut out on the bottom end 111 of the frontward facing side 106 and the bottom end 111 of the rearward facing side 107. Each triangular shaped cut out corresponds in cross-sectional configuration to a first end 153 of a diamond shaped protrusion 152 located on a frontward facing side 131 and a rearward facing side 133 of a first housing 130, as will be discussed. In operation, when the mouthpiece 105 is attached to the first housing 130, the first end 153 of each diamond shaped protrusion 152 on the first housing is tightly secured about each triangular shaped cut out on the mouthpiece.

The mouthpiece 105 includes at least one mouthpiece inlet 110(a), 110(b) on the mouthpiece sidewall 115(a), 115(b), and an air outlet 120 at a mouthpiece end 125. The at least one mouthpiece inlet 110(a), 110(b) is configured to increase the flow of air through the modular system 100 to produce smoother flavor and increased vapor production. In operation, when the user draws from the air outlet 120 of the mouthpiece 105, air flows from an outside environment through the at least one mouthpiece inlet 110(a), 110(b) and at least one second housing inlet 220, through at least one coil unit passageway 180 and to a tube 135, as will be discussed. A draw is a term used to refer to the inhalation of vapor from the mouthpiece 105. When there is an increase of air flowing through a coil unit, the vapor gets less dense and produces large clouds of vapor. Due to the highly dense water content in vapor, the vapor that is exhaled by the user is often referred to as clouds. This also means that because the vapor is less concentrated, the impact of the vapor on the user's throat is reduced, thereby providing a smoother taste. In addition, the increase of air flowing through the modular system 100 produces cooler vapor and offers a much looser, airier draw, like sucking on a wide straw.

In the present embodiment, the mouthpiece 105 contains one mouthpiece inlet 110(a), 110(b) on the bottom end 111 of each sidewall 115(a), 115(b). Each mouthpiece inlet 110(a), 110(b) has a rectangular shaped opening with a predetermined length and width. It should be appreciated that the mouthpiece inlet can have a variety of cross-sectional shapes and configurations and can be manufactured from a variety of different processes such as punching, stamping, scissoring, flame cutting, laser cutting, sawing, drilling, milling, or turning. The mouthpiece 105 may be comprised of any suitable material or combination of materials. Examples of suitable materials include metals, alloys, plastics or composite materials containing one or more of those materials, or thermoplastics that are suitable for food or pharmaceutical applications, for example polypropylene, polyetheretherketone (PEEK), ceramic, and polyethylene.

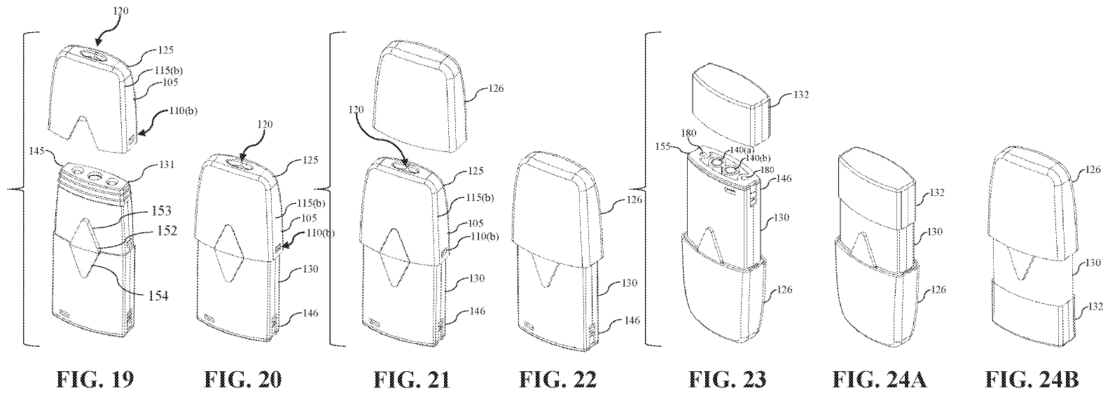

In another embodiment, as illustrated in FIGS. 19-22, the mouthpiece 105 includes a mouthpiece cover 126 that is configured to prevent dirt and debris from entering into the air outlet 120 located on the mouthpiece end 125 and to increase the durability and general life span of the mouthpiece 105. In the present embodiment, the mouthpiece cover is made of silicone and comprises a hollow square shaped body that is sized to fit snugly around the mouthpiece. It should be appreciated that the mouthpiece cover may be interchanged and may take additional forms, textures, ridges, and dimensions, and may be comprised of rubber materials such as neoprene, nitrile, and urethanes. The mouthpiece cover 126 can be manufactured from a variety of different processes such as injection molding, compression molding, and transfer molding.

The modular system 100 further includes a first housing 130 configured to removably attach to the mouthpiece 105. The first housing 130 comprises a hollow rectangular shaped body with a frontward facing side 131, a rearward facing side 133, two opposing sidewalls 134(a), 134(b), a first end 145, and a second end 146. However, shapes may also be used and are within the spirit and scope of the present invention. The first housing may be formed from the same material as the mouthpiece 105 or may be formed from another suitable material. In the present embodiment, the first housing includes a detent member 149(a), 149(b) located near the first end 145 of each sidewall 134(a), 134(b), Each detent member is configured to engage each mouthpiece inlet 110(a), 110(b) located on the bottom end 111 of each mouthpiece sidewall 115(a), 115(b).

The first housing 130 further includes a detent opening 151(a), 151(b) located near the second end 146 of each sidewall 134(a), 134(b). Each detent opening is configured to receive a second housing detent member (not shown) located inside the second housing, as will be discussed. In another embodiment, the first housing 130 is permanently attached to the mouthpiece 105. It should be appreciated that other attachment devices can be used to removably or permanently attach the mouthpiece to the first housing, including screws, bolts, welds, pins, clamps, brackets, magnets, male-female interference-type connections, waterproof adhesives, or any other suitable method known in the art.

Additionally, the first housing 130 includes a diamond shaped protrusion 152 comprising a first end 153 and a second end 154. The diamond shaped protrusion is located on the center of the frontward facing side 131 and on the center of the rearward facing side 133 of the first housing. As previously mentioned, the first end of the diamond shaped protrusions corresponds in cross-sectional configuration to the triangular shaped cut outs located on the bottom end 111 of the frontward facing side 106 and rearward facing side 107 of the mouthpiece 105. The second end of the diamond shaped protrusion corresponds in cross-sectional configuration to a triangular shaped cut out located on a first end 210 of the frontward facing side 206 and rearward facing side 207 of the second housing 205. In operation, when the first housing 130 is attached to the second housing, the second end of the diamond shaped protrusions on the first housing 130 are tightly secured about the triangular shaped cut outs on the second housing.

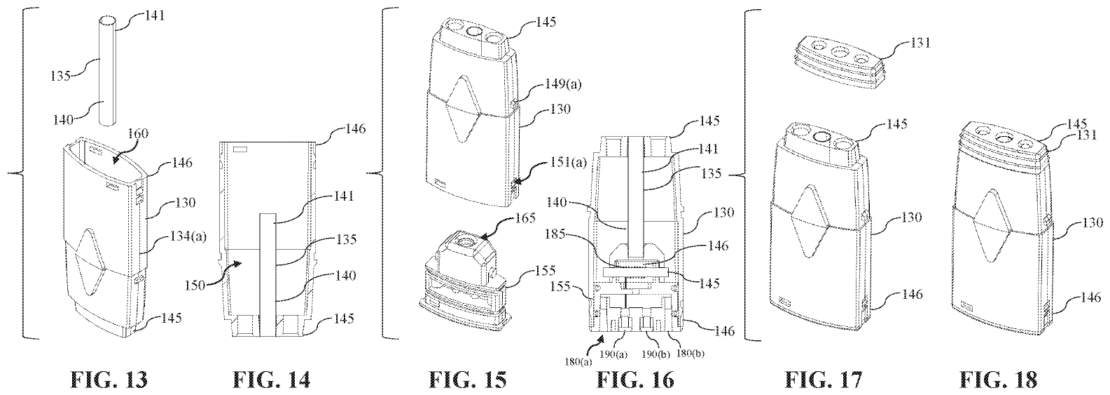

As shown in FIGS. 13 and 14, the first housing 130 includes a tube 135 that is disposed within the first housing 130. The tube has a hollow cylindrical shaped body comprising a first end 140 and a second end 141. The tube is configured such that the first end 140 of the tube is proximate to a first end 145 of the first housing 130, and the second end of the tube is proximate to a first end 170 of the coil unit 155. It should be appreciated that the tube can have a variety of cross-sectional shapes and configurations and may be formed from the same material as the first housing or may be formed from another suitable material.

An oil chamber 150 is located within the first housing. The oil chamber is configured for storing liquid vaporizing material. Liquid vaporizing material typically contains vegetable glycerin, propylene glycol, nicotine and flavoring. Alternatively, or in addition, liquid vaporizing material may include a nontobacco material. For example, liquid vaporizing material may include water, oils, solvents, ethanol, plant extracts and natural or artificial flavors. As best illustrated in FIGS. 7 and 8, a first housing sealing cap 131 attaches to the first end 145 of the first housing 130. When the modular system 100 is in the fully assembled configuration, the first housing sealing cap 131 prevents liquid vaporizing material within the oil chamber 150 from exiting the first end of the first housing.

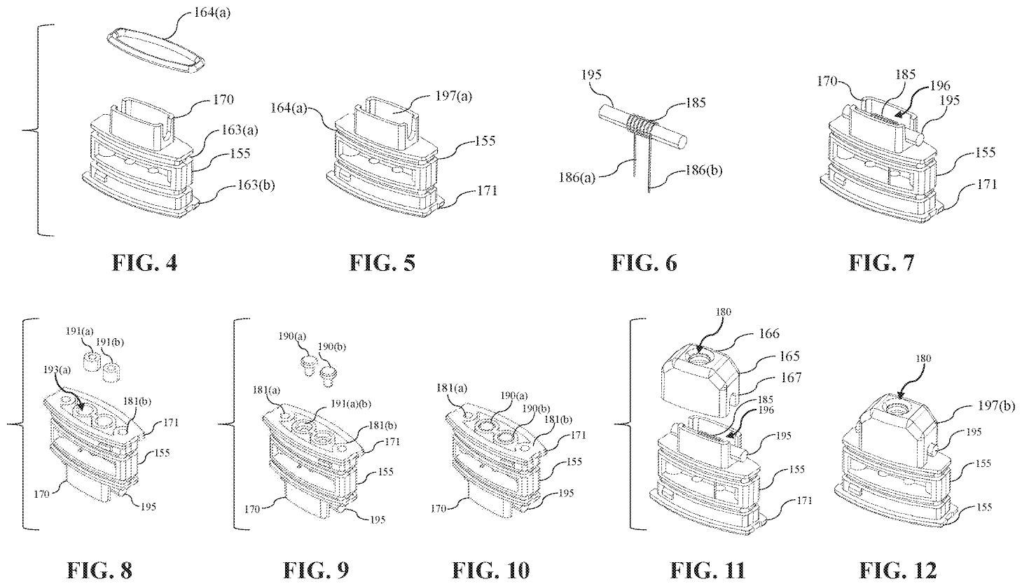

FIGS. 4-12 illustrate the assembly sequence of the coil unit and will be discussed together for ease of reference. The coil unit 155 is configured to be removably attachable within a coil unit compartment 160 of the first housing 130. In the present embodiment, the coil unit has a hollow rectangular shaped body comprising a first end 170 and a second end 171. The first end of the coil unit includes a first part 197(a) of a heating chamber 196, as will be discussed. It should be appreciated that the coil unit may take additional forms and dimensions and may be formed from the same material as the first housing or may be formed from another suitable material.

As illustrated in FIGS. 4 and 5, the coil unit employs a sliding fit sealed by a pair of silicone rings 164(a), 164(b) such that the coil unit can be easily fitted or removed from the coil unit compartment 160 of the first housing 130. The silicone rings 164(a), 164(b) are wrapped around a pair of circumferential outer grooves 163(a), 163(b) located on an exterior surface of the coil unit. It should be appreciated that the silicone rings may be interchanged and may take additional forms, textures, ridges, and dimensions, and may be formed from nitrile or other synthetic rubber-like materials. In another embodiment, the coil unit is permanently attached to the coil unit compartment of the first housing 130.

As illustrated in FIGS. 11 and 12, a mouth 165 is located at the first end 170 of the coil unit 155. The mouth 165 of the coil unit is configured for receiving the second end 141 of the tube 135 such that vapor, and air can pass through the mouth and into the tube when the system 100 is in the fully assembled configuration. In the present embodiment, the mouth comprises a hollow rectangular shaped body having a top end 166 and a bottom end 167. The top end of the mouth includes a circular shaped opening 180 that is configured for receiving the second end 141 of the tube 135. The bottom end of the mouth 165 includes a second part 197(b) of the heating chamber 196. The heating chamber houses a heating element and a wick, such that when heated produces vapor, as will be discussed. It should be appreciated that the mouth may be interchanged and may take additional forms, textures, ridges, and dimensions, and may be formed from nitrite or other synthetic rubber-like materials.

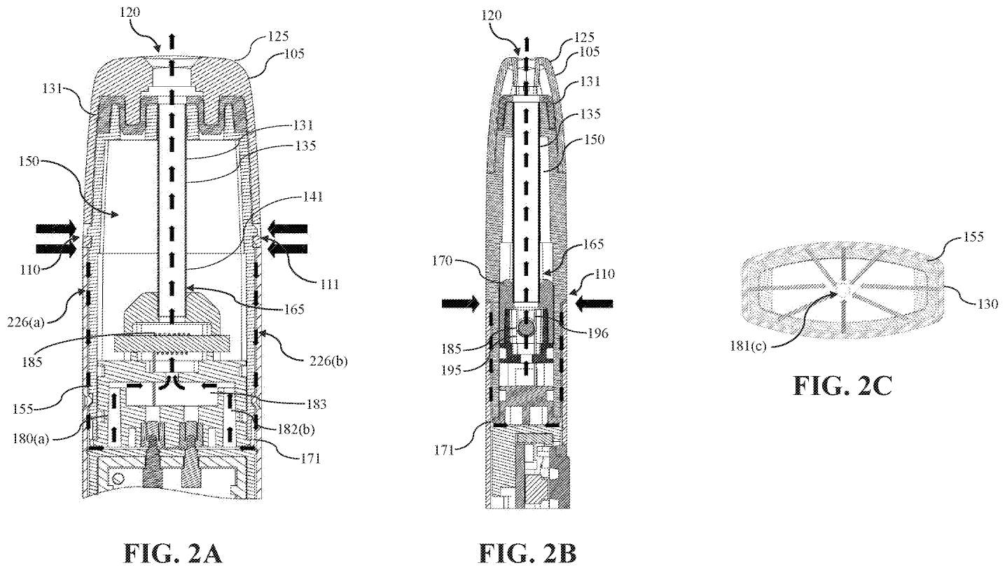

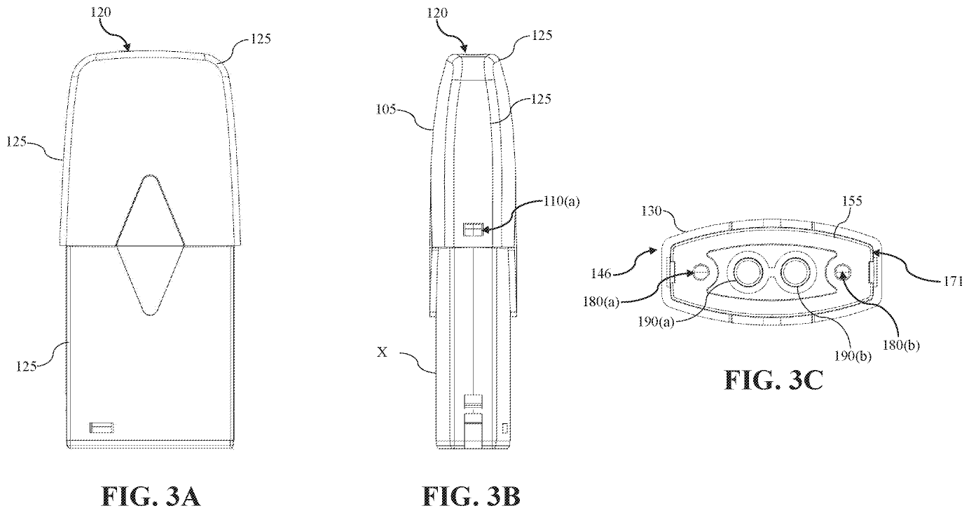

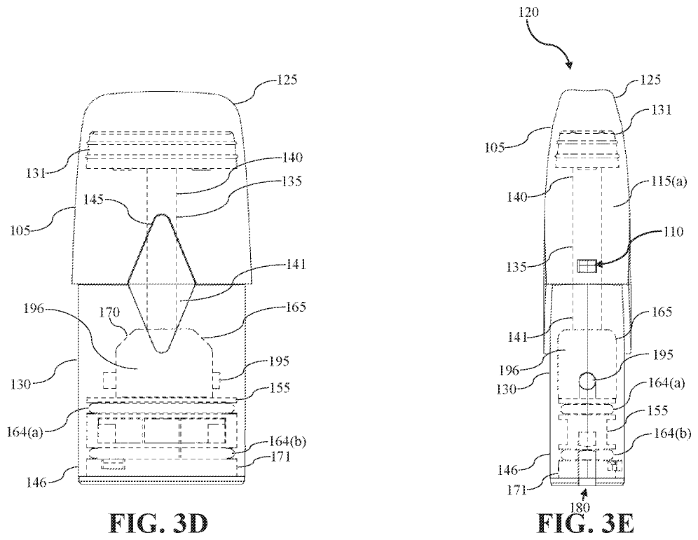

At least one coil unit passageway 180(a), 180(b) is located within the coil unit. In the present embodiment, the coil unit includes two coil unit passageways, each comprising a circular shaped opening 181(a), 181(b) and a vertical air passage 182(a), 182(b). Each vertical air passage which opens into an interior of the coil unit that defines a central channel 183. As best illustrated in FIG. 2C, the central channel 183 comprises a third opening 181(c) that is configured to allow air to flow inside the heating chamber. When there is an increase of air flowing through the coil unit, the vapor gets less dense and produces large clouds of vapor. This also means that because the vapor is less concentrated, the impact of the vapor on the user's throat is reduced, thereby providing a smoother taste. In addition, the increase of air flowing through the modular system 100 produces cooler vapor and offers a much looser, airier draw, like sucking on a wide straw.

A heating element 185 is located near the first end 170 of the coil unit 155. The heating element is configured to vaporize the liquid vaporizing material inside the oil chamber 150. In the present embodiment, as illustrated in FIGS. 11 and 12, the heating element is comprised of a resistance wire shaped into a coil having a first end 186(a) and a second end 186(b). It should be appreciated that the resistance wire can have additional forms and dimensions, and may be comprised of materials such as kanthal, nichrome, and nickel.

The heating element is in contact with a wick 195. The wick delivers the liquid vaporizing material to the heating element by capillary action. In the present embodiment, as shown in FIG. 6, the heating element is coiled around the wick and is positioned inside the heating chamber 196. The wick protrudes outwardly through a pair of openings located on the heating chamber into opposite sides of the oil chamber for contact with the liquid vaporizing material contained therein. When electricity from a power source is applied, the heating element vaporizes the liquid vaporizing material. The wick may be comprised of any suitable material or combination of materials. Examples of suitable materials include cotton, twisted silica cord, ceramic, and stainless-steel mesh.

As different liquid vaporizing materials have different properties when used in different coils, in different wicks, and in different air flow configurations, part of the functionality and attraction of the present invention is that it includes interchangeable parts to enable users to modify the character of the modular system 100 and experiment with various arrangements. For example, although many coil units use a cotton wick as their heating element, the user may prefer to use a coil unit containing a ceramic wick because it can withstand the high temperatures needed to vaporize cannabis oil without burning the material. Additionally, the use of interchangeable parts allows the user to clean and refill the oil chamber with a variety of liquid vaporizing materials by simply removing the coil unit from the coil unit compartment of the first housing.

A pair of first conductors 190(a), 190(b) defines a first contact surface positioned proximate to a second end 171 of the coil unit 155. The first conductors are configured for providing electrical communication between the heating element 185 and a power source externally located from the coil unit. In the present embodiment, each first conductor 190(a), 190(b) is wrapped by a sealing silicone sleeve 191(a), 191(b) and inserted into a first conductor opening 193(a), 193(b). A first end 186 of the heating element 185 is in electrical communication with one of the first conductors 190(a) and a second end 187 of the heating element is in electrical communication with the other first conductor 190(b). Preferably, the first conductors 190(a), 190(b) are highly conductive and temperature resistant and the heating element is highly resistive so that heat generation occurs primarily along the coils the heating element inside the heating chamber.

In another embodiment, as illustrated in FIGS. 23, 24A, and 24B, the first housing 130 includes a first housing cover 132 that is configured to prevent dirt and debris from entering into the coil unit openings located on the bottom end of the coil unit and to increase the durability and general life span of the first housing and coil unit. In the present embodiment, the first housing cover 132 is made of silicone and comprises a hollow rectangular shaped body that is sized to fit snugly around the first housing 130. It should be appreciated that the first housing cover may be interchanged and may take additional forms, textures, ridges, and dimensions and may be formed from the same material as the mouthpiece cover or may be formed from another suitable material.

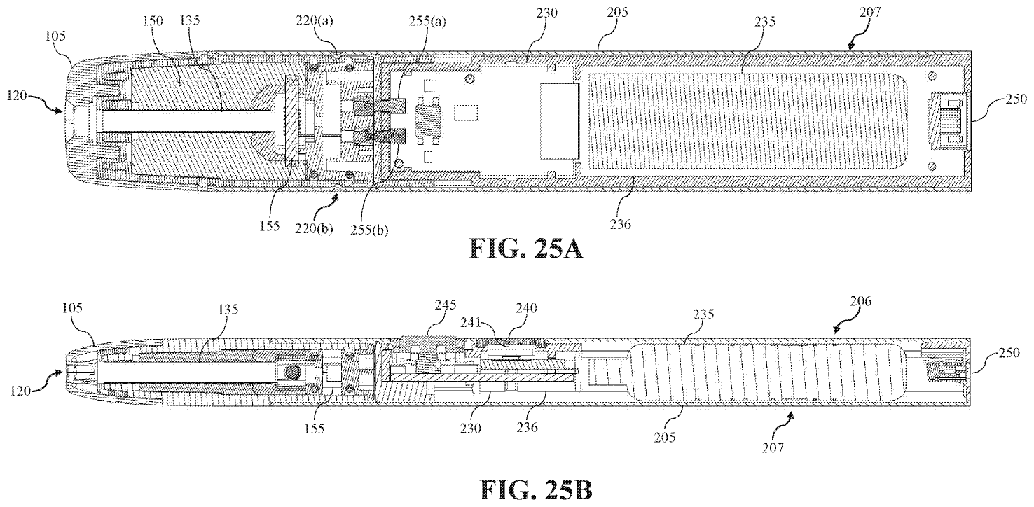

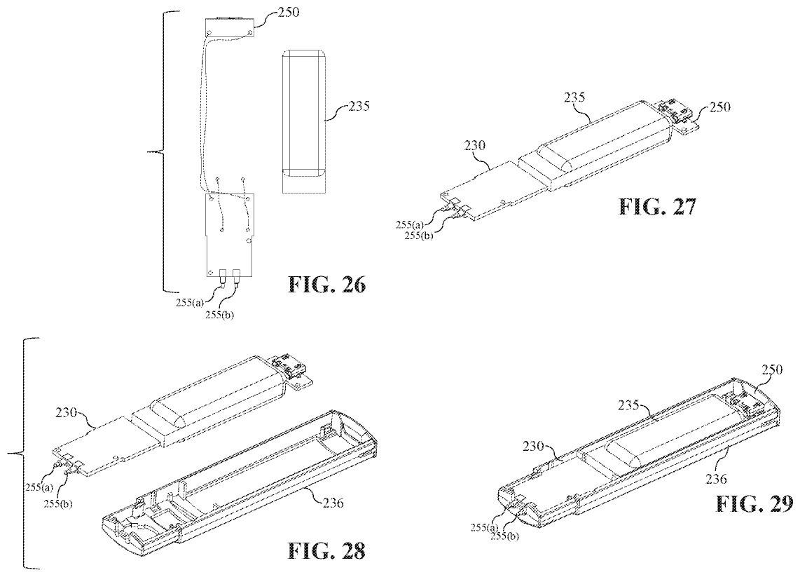

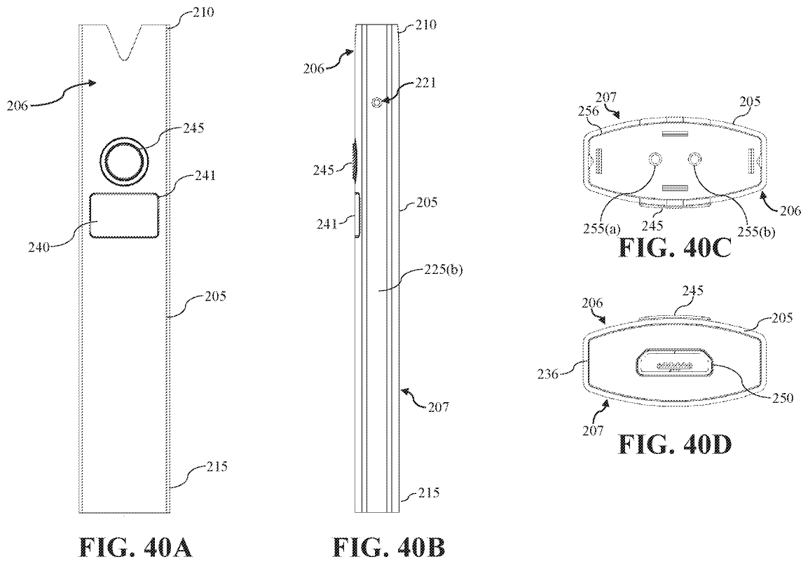

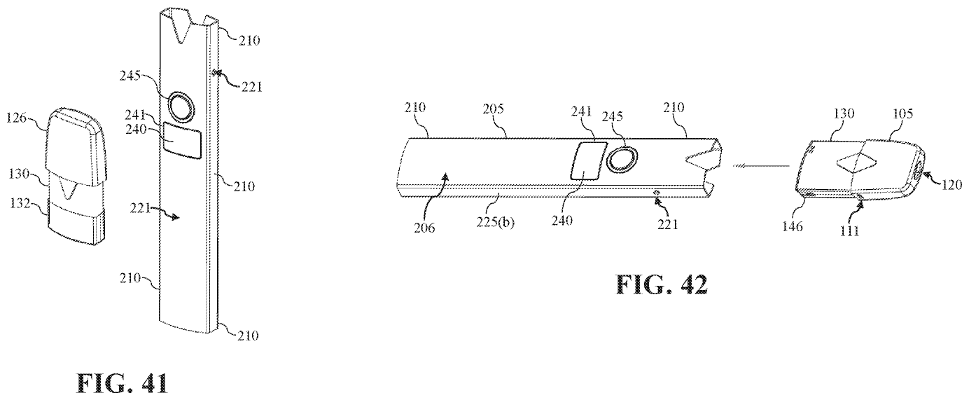

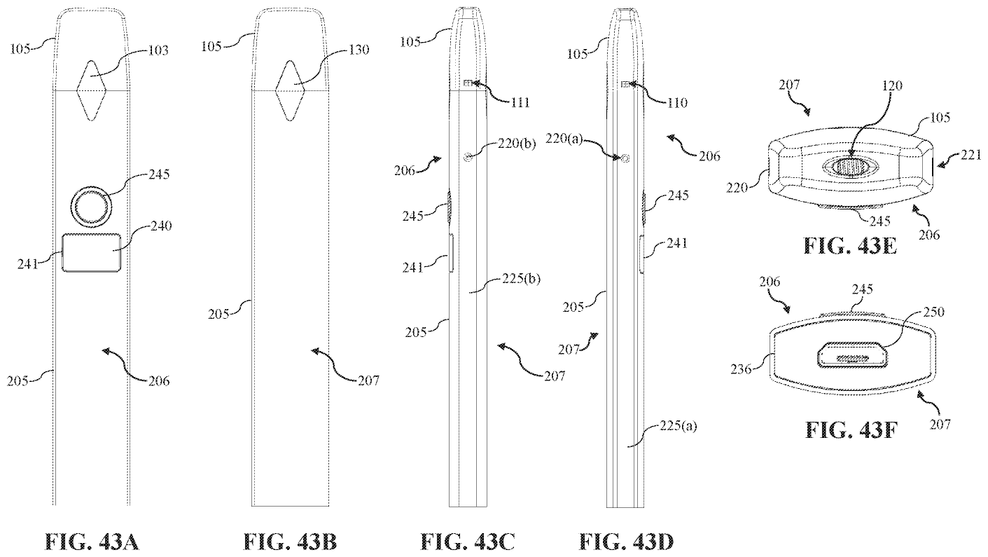

FIGS. 25-40D illustrate the assembly sequence of the second housing assembly and will be discussed together for ease of reference. The modular system 100 includes a second housing 205 having a first end 210 and a second end 215. The first end 210 of the second housing 205 is configured to removably attach with the second end 146 of the first housing 130. In other embodiments, the first housing and second housing are permanently attached. In the present embodiment, the second housing comprises a hollow rectangular shaped body with a frontward facing side 206, a rearward facing side 207, and two opposing sidewalls 225(a), 225(b). Each sidewall 225(a), 225(b) inside the first end 145 of the second housing includes a detent member (not shown). The detent members are configured to engage the first housing detent openings 151(a), 151(b) located near the second end 146 of each sidewall 134(a), 134(b). It should be appreciated that other attachment devices can be used to secure the first housing to the second housing, including screws, bolts, welds, pins, clamps, brackets, magnets, male-female interference-type connections, waterproof adhesives, or any other suitable method known in the art.

The second housing also includes a triangular shaped cut out on the first end 210 of the frontward facing side 206 and the first end of the rearward facing side 207. As previously mentioned, the triangular shaped cut out corresponds in cross-sectional configuration to the second end of the diamond shaped protrusion located on the frontward facing side 131 and the rearward facing side 133 of the first housing. In operation, when the first housing is attached to the second housing, the second end of the diamond shaped protrusions on the first housing are tightly secured about the triangular shaped cut outs on the second housing.

The second housing also contains at least one second housing inlet 220(a), 220(b) on the first end 210 of each sidewall 225(a), 225(b). In the present embodiment, each second housing inlet has a circular shaped opening with a predetermined cross-sectional area. It should be appreciated that the second housing inlet can have a variety of cross-sectional shapes and configurations and can be manufactured from a variety of different processes such as punching, stamping, scissoring, flame cutting, laser cutting, sawing, drilling, milling, or turning. The second housing may be formed from the same material as the first housing or may be formed from another suitable material.

In the fully assembled configuration, as illustrated in FIG. 2A, 2B, and 43A-43F, a pair of airflow channels 226(a), 226(b) are located between the interior sidewalls of the second housing and the exterior sidewalls of the first housing. Each airflow channel 226(a), 226(b) is configured to transport air flow from each mouthpiece inlet 110(a), 110(b) and each second housing inlet 220(a), 220(b), through at least one coil unit passageway 180 and to the tube 135. When there is an increase of air flowing through the coil unit, the vapor gets less dense and produces large clouds of vapor. This also means that because the vapor is less concentrated, the impact of the vapor on the user's throat is reduced, thereby providing a smoother taste. In addition, the increase of air flowing through the modular system 100 produces cooler vapor and offers a much looser, airier draw, like sucking on a wide straw.

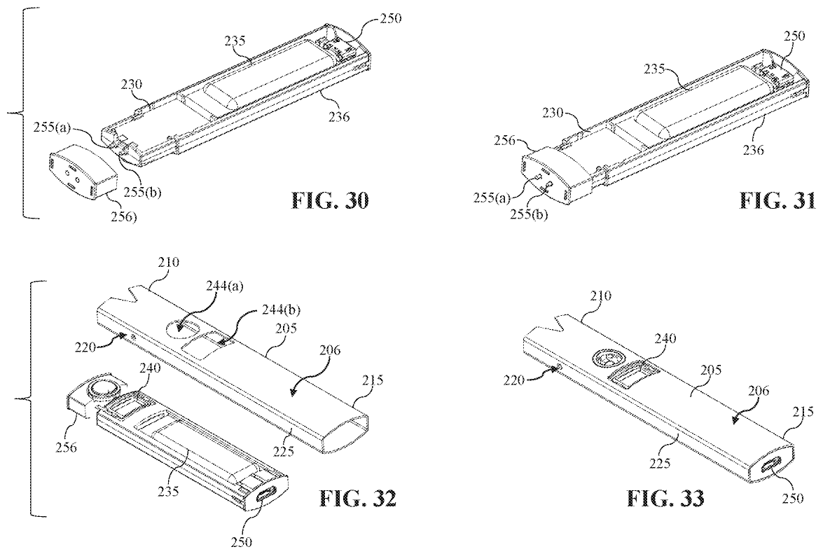

As illustrated in FIGS. 30 and 31, the second housing includes a second housing sealing cap 256 that is configured to prevent dirt and debris from entering the first end 145 of the second housing. In the present embodiment, the silicone end cap 256 comprises a hollow rectangular shaped body that is sized to fit snugly inside the first end 145 of the second housing. It should be appreciated that the silicone end cap 256 may take additional forms, textures, ridges, and dimensions and may be formed from nitrile or other synthetic rubber-like materials.

A central processing unit 230 containing the operating logic and software instructions for the modular system 100 is located inside the second housing. The central processing unit is conductively and communicatively coupled with a power source 235 located inside the second housing. In the present embodiment, the power source 235, is a rechargeable lithium battery due to its high voltage and high charge density. It should be appreciated that other types of rechargeable batteries or multiple batteries and/or battery types can be used as a power source. A charging port 250 is positioned on the second end 215 of the second housing for recharging the power source. In the present embodiment, the charging port is a USB connector. As illustrated in FIGS. 30-32, the central processing unit, power source, and charging port are cradled in an inner frame 236 that is configured to slide into the second housing.

In another embodiment, the second housing includes at least one light emitting element 231 that is configured to emit light from the second housing. The at least one light emitting element is configured to light up when the modular system 100 is in use and may indicate the charge state of the power source by flashing the at least one light emitting element in various colors or patterns. The at least one light emitting element is capable of emitting light of more than one color. Exemplary color-changing lights are RGB LEDs, bi-color LEDs, flashing LEDs, digital RGB LEDs, (organic) OLEDs, and RGB WLEDs (White LEDs).

The second housing further includes a display 240 that is conductively and communicatively coupled with the central processing unit and power source. The display is configured to display a graphical representation of at least an amount of charge in the power source and a voltage at which power is provided from the power source. In the present embodiment, as best illustrated in FIGS. 38-39, the display includes a display cover 241 positioned inside a first slot 244(a) on the frontward facing side of the second housing such that the display cover is flush with the frontward facing surface of the second housing.

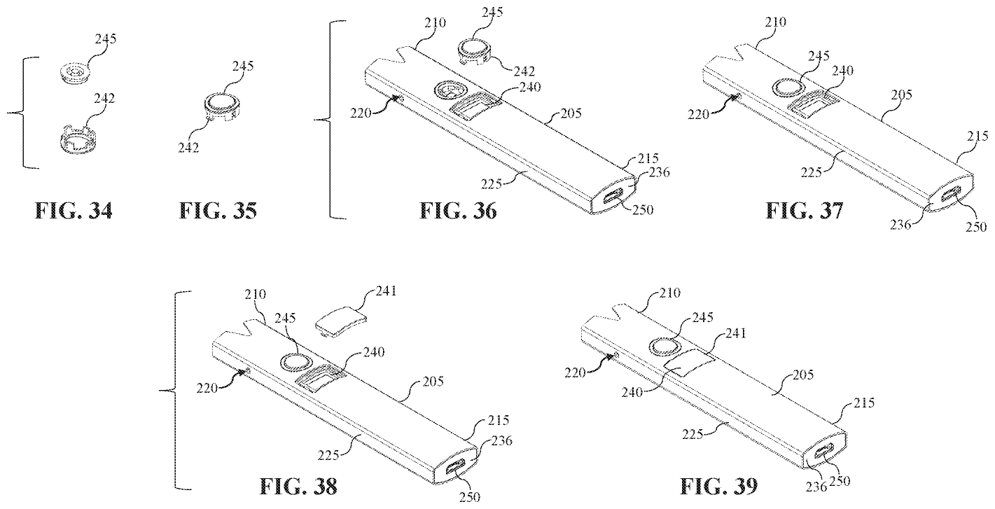

The second housing includes at least one control 245 for controlling the modular system 100. In the present embodiment, the control comprises a button that is conductively and communicatively coupled with the central processing unit and the power source. The button can be depressed or pushed a predetermined number of times to control the functionality of the modular system 100, including the voltage at which power is provided from the power source. As best illustrated in FIGS. 34-37, the button is secured to a button holder 242. The button holder is positioned inside a second slot 244(b) on the frontward facing surface of the second housing such that the button is flush with the frontward facing surface of the second housing.

A pair of second conductors 255(a), 255(b) are located inside the first end 145 of the second housing. Each second conductor comprises an elongated shaped element and is configured to contact and provide electrical communication with the pair of first conductors 190(a), 190(b) located on the second end 171 of the coil unit 155. Preferably, the second conductors 255(a), 255(b) are highly conductive and temperature resistant.

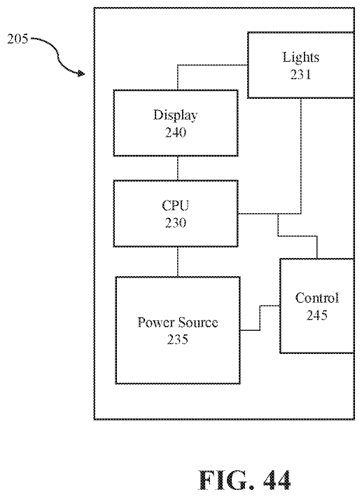

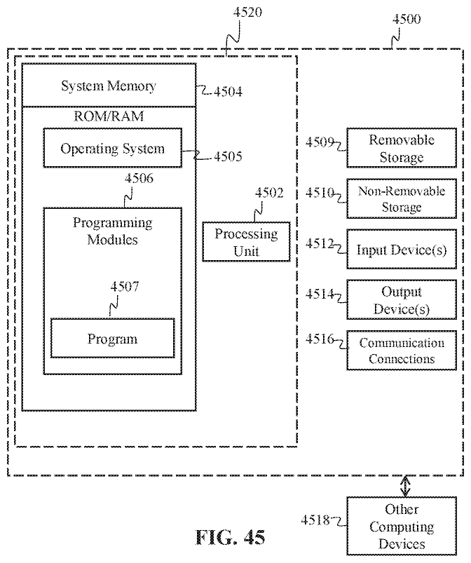

FIG. 5 is a block diagram of a system including an example computing device or central processing unit 4500 and other computing devices. Consistent with the embodiments described herein, the aforementioned actions performed by central control unit or central processing unit 230 may be implemented in a computing device, such as the computing device 4500 of FIG. 45. Any suitable combination of hardware, software, or firmware may be used to implement the computing device 4500. The aforementioned system, device, and processors are examples and other systems, devices, and processors may comprise the aforementioned computing device. Furthermore, computing device 500 may comprise an operating environment for central processing unit in FIG. 44 above.

With reference to FIG. 45, a system consistent embodiment of the invention may include a plurality of computing devices, such as computing device 4500. In a basic configuration, computing device d500 may include at least one processing unit 4502 and a system memory 4504. Depending on the configuration and type of computing device, system memory 4504 may comprise, but is not limited to, volatile (e.g. random access memory (RAM)), non-volatile read-only memory (ROM)), flash memory, or any combination or memory. System memory 4504 may include operating system 4505, one or more programming modules 4506 (such as program module 507). Operating system 4505, for example, may be suitable for controlling computing device 4500's operation. In one embodiment, programming modules 4506 may include, for example, a program module 4507. Furthermore, embodiments of the invention may be practiced in conjunction with a graphics library, other operating systems, or any other application program and is not limited to any particular application or system. This basic configuration is illustrated in FIG. 45 by those components within a dashed line 4520.

Computing device 4500 may have additional features or functionality. For example, computing device 4500 may also include additional data storage devices (removable and/or non-removable) such as, for example, magnetic disks, optical disks, or tape. Such additional storage is illustrated in FIG. 45 by a removable storage 4509 and a non-removable storage 4510. Computer storage media may include volatile and nonvolatile, removable and non-removable media implemented in any method or technology for storage of information, such as computer readable instructions, data structures, program modules, or other data. System memory 4504, removable storage 4509, and non-removable storage 4510 are all computer storage media examples (i.e. memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 4500. Any such computer storage media may be part of device 4500. Computing device 4500 may also have input device(s) 4512 such as a keyboard, a mouse, a pen, a sound input device, a camera, a touch input device, etc. Output device(s) 4514 such as a display, speakers, a printer, etc. may also be included. The aforementioned devices are only examples, and other devices may be added or substituted.

Computing device 4500 may also contain a communication connection 4516 that may allow device 4500 to communicate with other computing devices 4518, such as over a network in a distributed computing environment, for example, an into or the Internet. Communication connection 4516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data tiles may be stored in system memory 4504, including operating system 4505. While executing on processing unit 4502, programming modules 4506 may perform processes including, for example, one or functions of the above. Computing device 4502 may also include a graphics processing unit 4503, which supplements the processing capabilities of processor 4502 and which may execute programming modules 4506, including all or a portion of those processes and methods for the device explained above. The aforementioned processes are examples, and processing units 4502, 4503 may perform other processes. Other programming modules that may be used in accordance with embodiments of the present invention may include electronic mail and contacts applications, word processing applications, spreadsheet applications, database applications, slide presentation applications, drawing or computer-aided application programs, etc.

Generally, consistent with embodiments of the invention, program modules may include routines, programs, components, data structures, and other types of structures that may perform particular tasks or that may implement particular abstract data types. Moreover, embodiments of the invention may be practiced with other computer system configurations, including hand-held devices, multiprocessor systems, microprocessor-based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general purpose computer or in any other circuits or systems.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the invention have been described, other embodiments may exist. Furthermore, although embodiments of the present invention have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A modular system for vaporizing liquid vaporizing material comprising:
a mouthpiece having at least one mouthpiece inlet on a mouthpiece sidewall and an air outlet at a mouthpiece end;
a first housing configured to removably attach to the mouthpiece, and wherein the first housing comprises,
a tube disposed within the first housing such that a first end of the tube is proximate to a first end of the first housing,
an oil chamber within the first housing, the oil chamber for storing liquid vaporizing material,
a coil unit configured to be removably attachable within a coil unit compartment of the first housing, the coil unit comprising,
a mouth at a first end of the coil unit,
at least one coil unit passageway within the coil unit,
a heating element,
a pair of first conductors for providing electrical communication between the heating element and a power source externally located from the coil unit,
a wick in contact with the heating element and positioned with a heating chamber; and,
a second housing having a first end and a second end and having at least one second housing inlet on a second housing sidewall, wherein the first end of the second housing is configured to removably attach with a second end of the first housing, the second housing carrying
a central processing unit,
the power source,
a display,
at least one control for controlling the system,
a charging port and,
a pair of second conductors configured to contact and provide electrical communication with the first set of conductors.

2. The modular system of claim 1, wherein the mouth of the first end of the coil unit is configured for receiving a second end of the tube such that vapor, and air can pass through the mouth and into the tube when the system is in the fully assembled configuration.

3. The modular system of claim 1, wherein each first conductor defines a first contact surface positioned proximate to a second end of the coil unit.

4. The modular system of claim 1, wherein each second conductor comprises an elongated shaped element configured to contact one of the first conductors.

5. The modular system of claim 1, wherein in the heating element is coiled around the wick and a first end of the heating element is in electrical communication with one of the first conductors and a second end of the heating element is in electrical communication with the other first conductor.

6. The modular system of claim 1, wherein in the fully assembled configuration air flows from an outside environment through at least one of the mouthpiece inlet and second housing inlet, through the at least one coil unit passageway and to the tube.

7. The modular system of claim 1, wherein in the fully assembled configuration a first housing sealing cap attaches to the first end of the first housing and prevents oil within the oil chamber from exiting the first end of the first housing.

8. The modular system of claim 1, wherein the display is configured to display a graphical representation of at least an amount of charge in the power source and a voltage at which power is provided from the power source.

9. A modular system for vaporizing liquid vaporizing material comprising:
a mouthpiece having at least one mouthpiece inlet on a mouthpiece sidewall and an air outlet at a mouthpiece end;
a first housing configured to removably attach to the mouthpiece, and wherein the first housing comprises,
a tube disposed within the first housing such that a first end of the tube is proximate to a first end of the first housing,
an oil chamber within the first housing, the oil chamber for storing liquid vaporizing material,
a coil unit configured to be removably attachable within a coil unit compartment of the first housing, the coil unit comprising,
a mouth at a first end of the coil unit configured for receiving a second end of the tube such that vapor, and air can pass through the mouth and into the tube when the system is in the fully assembled configuration,
at least one coil unit passageway within the coil unit,
a heating element,
a pair of first conductors for providing electrical communication between the heating element and a power source externally located from the coil unit, wherein each first conductor defines a first planar contact surface positioned proximate to the second end of the coil unit,
a wick in contact with the heating element and positioned with a heating chamber; and,
a second housing having a first end and a second end and having at least one second housing inlet on a second housing sidewall, wherein the first end of the second housing is configured to removably attach with a second end of the first housing, the second housing carrying,
a central processing unit,
the power source,
a display,
at least one control for controlling the system,
a charging port and,
a pair of second conductors configured to contact and provide electrical communication with the first set of conductors, wherein each second conductor comprises an elongated shaped element configured to contact one of the first conductors.

10. The modular system of claim 9, wherein each second conductor comprises an elongated shaped element configured to contact one of the first conductors.

11. The modular system of claim 9, wherein in the heating element is coiled around the wick and a first end of the heating element is in electrical communication with one of the first conductors and a second end of the heating element is in electrical communication with the other first conductor.

12. The modular system of claim 9, wherein in the fully assembled configuration air flows from an outside environment through at least one of the mouthpiece inlet and second housing inlet, through the at least one coil unit passageway and to the tube.

13. The modular system of claim 9, wherein in the fully assembled configuration a first housing sealing cap attaches to the first end of the first housing and prevents oil within the oil chamber from exiting the first end of the first housing.

14. The modular system of claim 9, wherein the display is configured to display a graphical representation of at least an amount of charge in the power source and a voltage at which power is provided from the power source.

15. A modular system for vaporizing liquid vaporizing material comprising:
a mouthpiece having an air outlet at a mouthpiece end;
a first housing attached to the mouthpiece, and wherein the first housing comprises, a tube disposed within the first housing and an oil chamber for storing liquid vaporizing material;
a coil unit within a coil unit compartment of first housing, the coil unit comprising,
a mouth at a first end of the coil unit,
at least one coil unit passageway within the coil unit,
a heating element,
a pair of first conductors for providing electrical communication between the heating element and a power source externally located from the coil unit, and,
a wick in contact with the heating element and positioned with a heating chamber; and,
a second housing configured to removably attach with a second end of the first housing, the second housing carrying,
a central processing unit,
the power source,
a display,
at least one control for controlling the system,
a charging port, and
a pair of second conductors configured to contact and provide electrical communication with the first set of conductors.

16. The modular system of claim 16, wherein the mouth of the first end of the coil unit is configured for receiving a second end of the tube such that vapor, and air can pass through the mouth and into the tube when the system is in the fully assembled configuration,

17. The modular system of claim 16, wherein each first conductor defines a first contact planar surface positioned proximate to the second end of the coil unit.

18. The modular system of claim 16, wherein each second conductor comprises an elongated shaped element configured to contact one of first planar contact surface of the first conductors.

19. The modular system of claim 16, wherein in the fully assembled configuration air flows from an outside environment through the at least one coil unit passageway and to the tube.
